(19) United States
(12) Patent Application Publication
(10) Pub. No.: US 2015/0259641 A1
(43) Pub. Date: Sep. 17, 2015

(54) GELATINOUS SUBSTANCE BASED GROWTH MEDIUM FILTRATION DEVICE AND METHOD

(71) Applicant: TOKU-E COMPANY, Bellingham, WA (US)

(72) Inventors: Qiubao Pan, Singapore (SG); Yohanes Permana Rusli, Shanghai (CN)

(21) Appl. No.: 14/664,625

(22) Filed: Mar. 20, 2015

Related U.S. Application Data
(62) Division of application No. 13/311,154, filed on Dec. 5, 2011, now abandoned.
(60) Provisional application No. 61/420,469, filed on Dec. 7, 2010.

(30) Foreign Application Priority Data
Oct. 21, 2011 (US) PCT/US2011/057259

(51) Int. Cl. C12N 1/00; B01D 61/02

(52) U.S. Cl. CPC C12N 1/00; B01D 61/025; B01D 2317/025

(57) ABSTRACT

Growth medium filtration devices are provided which are particularly well adapted to sterilize gelatinous substance based growth mediums including agar, agarose and other polysaccharide derived growth mediums for use in agar plates and the like for culturing microorganisms. The devices may include one or more filtration units and at least one heat source for filtering the growth medium in a heated filtration process. Methods of filtering a growth medium, such as an agar growth medium, and methods of preparing a growth medium having low gelling point and lower molecular weight characteristics are also provided.

### CROSS-REFERENCE TO RELATED APPLICATION

This application is a divisional of U.S. application Ser. No. 13/311,154, filed Dec. 5, 2011 which claims the benefit under 35 U.S.C. §119(e) of U.S. Provisional Patent Application No. 61/420,469, filed Dec. 7, 2010, and claims priority to Application No. PCT/US2011/057259, filed on Oct. 21, 2011, each of these applications are incorporated herein by reference in their entireties.

### BACKGROUND

1. Technical Field

This disclosure generally relates to devices and methods for filtering a growth medium used to culture microorganisms, and more particularly, to devices and methods for filter sterilizing gelatinous substance based growth mediums including agar, agarose and other polysaccharide derived growth mediums under elevated temperature conditions to be used in the preparation of agar plates and the like without the need for autoclave processes.

2. Description of the Related Art

Agar plates are traditionally prepared by subjecting the agar to autoclave sterilization or gamma sterilization processes. Both of these processes, however, can be time consuming and may damage nutrients in the agar.

For instance, the autoclave sterilization process typically includes preparation of the growth medium, autoclave heating time, sterilization, cooling and pouring of the agar plates. This process can therefore take about two to four hours depending on the sterilization requirements and the specific type of agar medium used in the process. Furthermore, when additives, such as, for example, bovine serum, antibiotics and vitamins are used, the instability of such additives can require even further processing steps after the autoclave sterilization process. In addition, the degree of uniformity of any additives in the prepared agar plates may be less than desired. The autoclave sterilization process may also cause nutritional loss in the agar growth medium and hence a reduction in the ability of the agar plate to grow certain microorganisms.

The gamma sterilization process generally takes days to prepare the agar plates and is very costly on a per item basis should only a small number of agar plates be required. As such, the gamma sterilization process is typically used with pre-poured agar when relatively large amounts of the same type of agar medium are used, as may be the case in certain hospital settings. Like the autoclave sterilization process, the gamma sterilization process can cause nutritional loss in the agar growth medium. In addition, when additives, such as, for example, bovine serum, antibiotics and vitamins are used, the instability of such additives can require even further processing steps after the gamma sterilization process, thus leading to even longer preparation time.

### BRIEF SUMMARY

Embodiments described herein provide gelatinous substance based growth medium filtration devices and methods which are particularly well adapted for sterilizing gelatinous substance based growth mediums for use in culturing microorganisms without the need for autoclave sterilization or gamma sterilization processes. According to some embodiments, the devices and methods provide particularly efficient means of processing agar, agarose, and other polysaccharide derived growth mediums for preparing agar plates and the like. In addition, the resultant sterilized growth medium is well adapted with respect to uniformity and nutritional content to culture microorganisms in a particularly effective manner.

According to one embodiment, a gelatinous substance based growth medium filtration device may be summarized as including an upstream filter unit configured to support an upstream membrane filter having a porosity to filter particles of a size greater than a first threshold size; a downstream filter unit configured to support a downstream membrane filter having a porosity to filter particles of a size greater than a second threshold size that is less than the first threshold size; a housing configured to receive the upstream filter unit and the downstream filter unit and support the upstream filter unit upstream of the downstream filter unit, and the housing including an internal cavity to receive the growth medium upstream of both of the upstream filter unit and the downstream filter unit; a pressurizing device coupled to the housing to selectively pressurize the internal cavity during a filtration operation to assist in forcing the growth medium through the upstream filter unit and the downstream filter unit sequentially; and a heat source coupled to the housing to maintain the growth medium at an elevated temperature within the internal cavity during at least a portion of the filtration operation.

The upstream filter unit and the downstream filter unit may each include a filter cartridge coupled to a membrane filter support, and wherein the filter units may be removably coupled to the housing to facilitate replacement of the upstream membrane filter and downstream membrane filter. The growth medium filtration device may further include seals positioned between the housing and each of the upstream and the downstream filter units to substantially prevent the growth medium from bypassing the upstream and the downstream membrane filters during the filtration operation. The upstream and the downstream filter units may be configured to position the upstream membrane filter offset from the downstream membrane filter and create a space between the upstream membrane filter and the downstream membrane filter to temporarily receive partially filtered growth medium during the filtration operation. The upstream and the downstream filter units may be configured to position the upstream membrane filter offset from the downstream membrane filter by at least one centimeter but less than or equal to ten centimeters. The housing may include a storage cavity positioned downstream of the filter units to receive the filtered growth medium after the growth medium passes through the filter units. The growth medium filtration device may further include a second heat source coupled to the housing to selectively maintain the filtered growth medium within the storage cavity at an elevated temperature. The growth medium filtration device may further include a relief valve coupled to the storage cavity to selectively relieve pressure within the storage cavity.

The first threshold size of the upstream membrane filter may be about 0.45 μm±0.03 μm and the second threshold size of the downstream membrane filter may be about 0.22 μm±0.03 μm. A ratio of the first threshold size of the upstream membrane filter to the second threshold size of the downstream membrane filter may be between 1.5 and 3.0. The first threshold size of the upstream membrane filter may be about twice the second threshold size of the downstream membrane filter. The heat source may be configured to maintain the temperature of the growth medium within the internal cavity at an elevated temperature at or above a gelling point of the growth medium or at or above a melting point of the growth medium. For example, in some embodiments, the heat source may be configured to maintain the temperature of the growth medium within the internal cavity at an elevated temperature at or above 45° C. during the filtration operation. The housing may include an upper housing assembly and a lower housing assembly removably coupled together. The upstream and the downstream filter units may be removably coupled between the upper housing assembly and the lower housing assembly. The growth medium filtration device may further include a discharge valve positioned at a lower end of the housing to selectively discharge filtered growth medium from the filtration device. The housing may include a funnel structure to direct the growth medium toward the upstream and the downstream filter units during operation. The growth medium filtration device may further include a stand coupled to the housing to position the housing above a support surface, the stand configured to provide a space beneath the housing sufficient to receive a Petri dish.

A method of filtering a growth medium may be summarized as including maintaining the growth medium at an elevated temperature within an internal cavity of a filtration device upstream of both of an upstream filter unit and a downstream filter unit, the upstream filter unit configured to support a upstream membrane filter having a porosity to filter particles of a size greater than a first threshold size and the downstream filter unit configured to support a downstream membrane filter having a porosity to filter particles of a size greater than a second threshold size that is smaller than the first threshold size; and pressurizing the internal cavity to assist in forcing the growth medium to pass sequentially through the upstream filter unit and the downstream filter unit to sterilize the growth medium for use in culturing microorganisms.

The growth medium may include maintaining an agar growth medium at an elevated temperature at or above a gelling point of the growth medium or at or above a melting point of the growth medium. For example, in some embodiments, the heat source may be configured to maintain the temperature of the growth medium within the internal cavity at an elevated temperature at or above 45° C. as the agar growth medium is forced to pass sequentially through the upstream and the downstream filter units. The agar growth medium may be a commercially available agar growth medium having a melting temperature at or above about 85° C. or may be an agar, agarose or other agar based growth medium having a relatively lower melting temperature in the range, for example, of about 45° C. to 85° C. The method may further include sealing an interface of each of the upstream and the downstream filter units to substantially prevent the growth medium from bypassing the upstream and the downstream membrane filters when pressurizing the internal cavity. The method may further include sealing the growth medium within the filtration device prior to pressurizing the internal cavity. The method may further include storing filtered growth medium in a storage cavity positioned downstream of both of the upstream and the downstream filter units. The method may further include maintaining the filtered growth medium at an elevated temperature within the storage cavity prior to discharging the filtered growth medium from the filtration device. The method may further include relieving pressure contained within the storage cavity. The method may further include coupling an upper housing assembly and a lower housing assembly of the filtration device together with the upstream and the downstream filter units received therebetween. The method may further include intermittingly discharging filtered growth medium from the filtration device into a series of Petri dishes. The method may further include filtering a source of an initial growth medium to extract a portion thereof which has a relatively lower gelling point and lower molecular weight characteristic and introducing the portion of the initial growth medium which has the relatively lower gelling point and lower molecular weight characteristic into the internal cavity of the filtration device as the growth medium to be filtered.

A method of preparing agar media powder may be summarized as including dissolving a dehydrated source of a nutrient agar with distilled water to form an agar nutrient solution; heating the agar nutrient solution to maintain the agar nutrient solution at an elevated temperature; filtering the heated agar nutrient solution to filter out relatively higher gelling point and higher molecular weight agar media particles; causing a refined source of agar media particles to separate from the filtered agar nutrient solution; collecting the refined source of agar media particles; and dehydrating the refined source of agar media particles to form an agar nutrient powder characterized by a relatively lower gelling point and lower molecular weight when the agar nutrient powder is combined with distilled water to form a gelatinous mass. Heating the agar nutrient solution may include heating the agar nutrient solution to between about 45° C. and 65° C. Filtering the heated agar nutrient solution may include passing the heated agar nutrient solution through at least one membrane filter which has a porosity to filter particles of a size greater than a first threshold size. Causing the refined source of agar media particles to separate from the filtered agar nutrient solution may include an ethanol precipitation process or polyethylene glycol precipitation process. Dehydrating the refined source of agar media particles to form the agar nutrient powder may include vacuum heating the refined source of agar media particles at a temperature between about 60° C. and 80° C. The method may further include storing the agar nutrient powder for subsequent use.

### BRIEF DESCRIPTION OF THE SEVERAL VIEWS OF THE DRAWINGS

FIG. 1 is an isometric view of a growth medium filtration device, according to one embodiment.

FIG. 2 is an exploded isometric view of the growth medium filtration device of FIG. 1.

FIG. 3 is a front elevational view of the growth medium filtration device of FIG. 1.

FIG. 4 is a cross-sectional view of the growth medium filtration device of FIG. 1 taken along line 4-4 of FIG. 3.

FIG. 5 is a process flow diagram of a method of preparing a refined source of growth media powder, according to one embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details. In other instances, well-known structures or steps associated with filtration equipment and filtration processes may not be shown or described in detail to avoid unnecessarily obscuring descriptions of the embodiments. For instance, it will be appreciated by those of ordinary skill in the relevant art that various clamping or fastening mechanisms other than those illustrated herein may be used to seal the growth medium filtration devices. As another example, it will be appreciated by those of ordinary skill in the relevant art that the devices can be configured to provide automated filtration devices and methods by incorporating an appropriate control system and monitoring system, including for example, systems including temperature and/or pressure sensors, flow control valves and other devices to monitor and control the performance of the filtration devices in an automatic or partially automatic manner.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

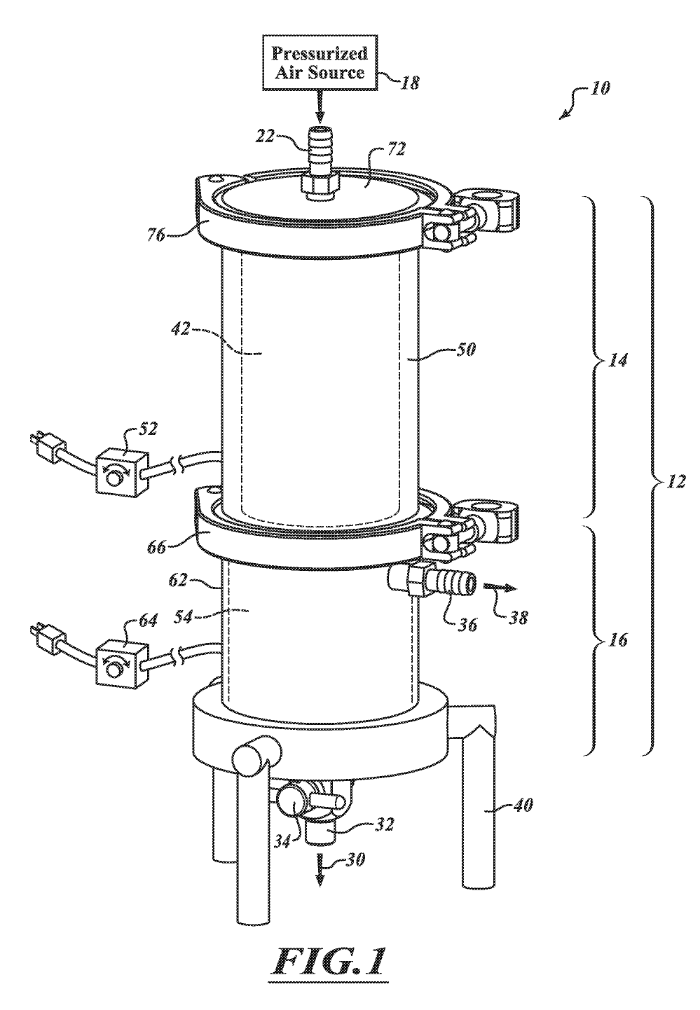

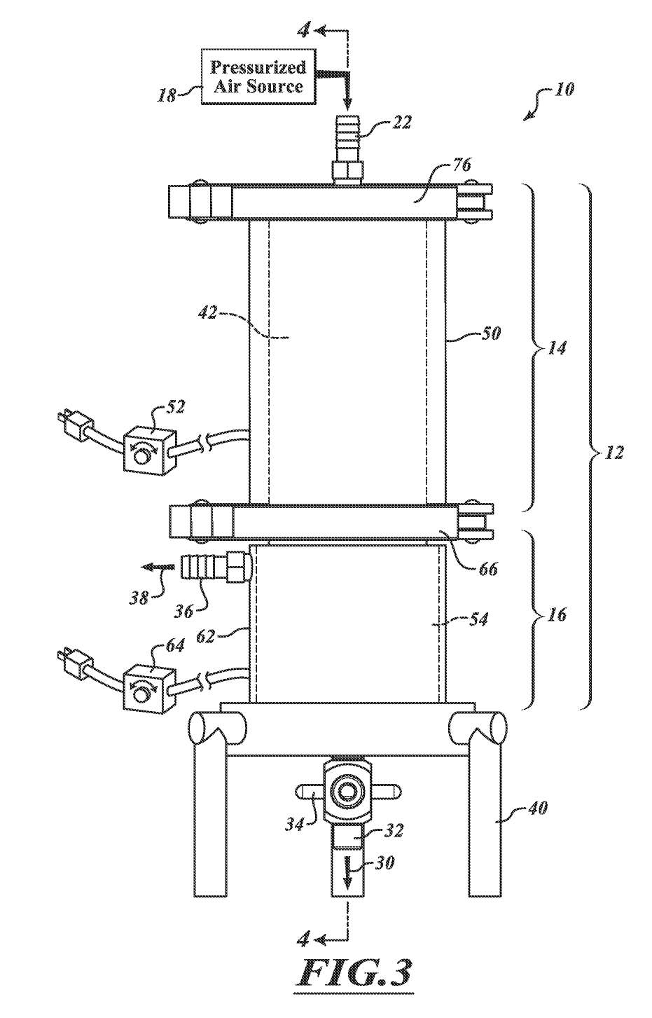

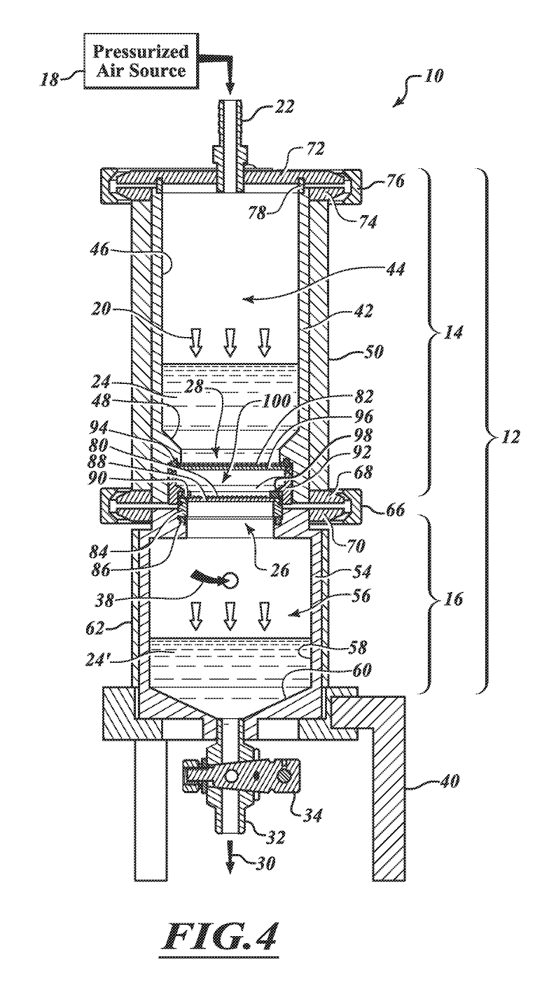

FIGS. 1 through 4 show a gelatinous substance based growth medium filtration device 10 according to one example embodiment. The filtration device 10 includes a housing 12 comprised of an upper housing assembly 14 and a lower housing assembly 16 coupled together. The housing 12 is configured to be attached to a pressurized air source 18, such as, for example, an air compressor, for pressurizing the filtration device 10 during a filtration operation as represented by the arrows labeled 20 in FIG. 4. The pressurized air source 18 may include an air filter device for filtering air prior to introduction into the housing 12. In addition, a pressure sensor or gauge may be provided along with appropriate valves and other control devices to selectively pressurize the housing 12 in a controllable and consistent manner. The pressurized air source 18 may be coupled to the housing 12 via a hose or other conduit (not shown) and a fitting 22. In some embodiments, the pressurized air source 18 may be configured to raise the pressure within the housing 12 from between 0.30 MPa and 0.75 MPa, and in some embodiments, the pressurized air source 18 may be configured to raise the pressure within the housing 12 to 0.40 MPa±0.10 MPa. In some embodiments, the pressurized air source 18 may be beneficially set at 0.40 MPa±0.05 MPa. In any event, the pressure setting is preferably balanced with respect to the viscosity of the material being filtered to filter the material in a steady, consistent and relatively efficient manner.

As best illustrated in FIG. 4, during the filtration process, a growth medium 24 for culturing microorganisms, such as, for example, an agar growth medium is forced to pass through a series of filter units 26, 28 to sterilize the growth medium 24. The growth medium may be a commercially available agar growth medium having a melting temperature at or above about 85° C. or may be an agar, agarose or other growth medium having a relatively lower melting temperature in the range, for example, of about 45° C. to 85° C. The sterilized growth medium 24′ may be temporarily stored in the housing 12 and subsequently discharged from the housing 12, as represented by the arrow labeled 30. More particularly, the housing 12 may include an outlet 32 having a discharge valve 34 for selectively discharging the sterilized growth medium 24′ from the filtration device 10. Pressure built-up within the housing 12 during the filtration process may be selectively vented through a relief valve (not shown) coupled to the housing 12 via a fitting 36, as represented by the arrow labeled 38.

A stand 40 may be provided to support the housing 12 at a height above a support surface on which the stand 40 may be situated. The stand 40 may include sufficient space to receive a Petri dish beneath the housing 12 to receive the sterilized growth medium 24′ from the outlet 32 of the filtration device 10. In some embodiments, the stand 40 may be in the form of a tripod, as shown in the figures.

As best illustrated in FIG. 4, the upper housing assembly 14 includes a main body portion 42 which defines an internal cavity 44 for receiving the growth medium 24 to be sterilized. The internal cavity 44 may be defined by a cylindrical sidewall 46 of the main body portion 42 and a funnel structure 48 at a lower end thereof. The funnel structure 48 is configured to funnel the growth medium 24 out of the upper housing assembly 14.

The main body portion 42 of the upper housing assembly 14 may be coupled to a heat source 50, such as, for example, an electric jacket heater, for enabling the contents within the internal cavity 44 of the upper housing assembly 14 to be selectively heated to and/or maintained at an elevated temperature prior to or during the filtration operation. Although the heat source 50 is illustrated as a distinct component of the filtration device 10, it is appreciated that the heat source 50 may be integrated into the main body portion 42 of the upper housing assembly 14.

In some embodiments, the heat source 50 is configured to provide sufficient heat to raise the temperature of the growth medium 24 within the internal cavity 44 to at or above a gelling point of the growth medium or at or above a melting point of the growth medium. For example, in some embodiments, the heat source 50 is configured to provide sufficient heat to raise the temperature of the growth medium 24 within the internal cavity 44 to 45° C. or more and/or maintain the growth medium 24 above 45° C. as the growth medium 24 is forced to pass sequentially through the filter units 26, 28. In other embodiments, the heat source 50 is configured to provide sufficient heat to raise the temperature of the growth medium 24 within the internal cavity 44 to 85° C. or more and/or maintain the growth medium 24 above 85° C. as the growth medium 24 is forced to pass sequentially through the filter units 26, 28. In still other embodiments, the heat source 50 may be configured to provide sufficient heat to raise and/or maintain the temperature of the growth medium 24 within the internal cavity 44 between about 85° C. and 100° C. as the growth medium is forced to pass sequentially through the filter units 26, 28. In still other embodiments, the heat source 50 is configured to provide sufficient heat to raise and/or maintain the temperature of the growth medium 24 within the internal cavity 44 between about 90° C. and 100° C. as the growth medium 24 is forced to pass sequentially through the filter units 26, 28. The heat source 50 may include a temperature regulation mechanism 52 and/or temperature sensing devices to regulate the temperature of the growth medium 24 within the internal cavity 44 of the main body portion 42 of the upper housing assembly 14 during operation.

As best illustrated in FIG. 4, the lower housing assembly 16 includes a main body portion 54 which defines an internal cavity 56 for receiving the sterilized growth medium 24′ after it is filtered. The internal cavity 56 may be defined by a cylindrical sidewall 58 of the main body portion 54 and a funnel structure 60 at a lower end thereof. The funnel structure 60 is configured to funnel the sterilized growth medium 24′ out of the lower housing assembly 16.

The main body portion 54 of the lower housing assembly 16 may be coupled to another heat source 62, such as, for example, another electric jacket heater, for enabling the contents within the internal cavity 56 of the lower housing assembly 16 to be selectively heated to and/or maintained at an elevated temperature. Although the heat source 62 is illustrated as a distinct component of the filtration device 10, it is appreciated that the heat source 62 may be integrated into the main body portion 54 of the lower housing assembly 16.

In some embodiments, the heat source 62 is configured to provide sufficient heat to raise and/or maintain the temperature of the sterilized growth medium 24′ within the internal cavity 56 at or above a gelling point of the growth medium 24′ or at or above a melting point of the growth medium 24′. For example, in some embodiments, the heat source 62 is configured to provide sufficient heat to raise and/or maintain the temperature of the growth medium 24′ within the internal cavity 56 at or above 45° C. prior to discharging the sterilized growth medium 24′ from the filtration device 10. In other embodiments, the heat source 62 is configured to provide sufficient heat to raise and/or maintain the temperature of the sterilized growth medium 24′ within the internal cavity 56 at or above 85° C. prior to discharging the sterilized growth medium 24′ from the filtration device 10. In still other embodiments, the heat source 62 is configured to provide sufficient heat to raise and/or maintain the temperature of the sterilized growth medium 24′ within the internal cavity 56 between about 85° C. and 100° C. prior to discharging the sterilized growth medium 24′ from the filtration device 10. In still other embodiments, the heat source 62 is configured to provide sufficient heat to raise and/or maintain the temperature of the sterilized growth medium 24′ within the internal cavity 56 between about 90° C. and 100° C. prior to discharging the sterilized growth medium 24′ from the filtration device 10. The heat source 62 may include a temperature regulation mechanism 64 and/or temperature sensing devices to regulate the temperature of the growth medium 24′ within the internal cavity 56 of the main body portion 54 of the lower housing assembly 16.

According to the example embodiment shown in FIGS. 1 through 4, the upper housing assembly 14 and lower housing assembly 16 are removably coupleable together. The housing assemblies 14, 16 may be coupled together with a clamping or fastening device 66, such as, for example, a quick release clamp mechanism. To facilitate coupling of the housing assemblies 14, 16 together, each of the housing assemblies 14, 16 may include a respective mounting flange 68, 70 sized and shaped to interface with the clamping or fastening device 66. The upper housing assembly 14 may further include a lid 72 coupleable to an upper mating flange 74 of the main body portion 42 via another clamping or fastening device 76, such as, for example, a quick release clamp mechanism. An annular seal 78 may be disposed between the lid 72 and the main body portion 42 of the upper housing assembly 14 to assist in maintaining a sealed environment within the housing 12 during operation of the filtration device 10.

The housing assemblies 14, 16 can be quickly decoupled and the internal cavities 44, 56 thereof accessed by releasing the clamping or fastening devices 66, 76. In this manner, the internal cavity 44 of the upper housing assembly 14 may be accessed to fill the cavity 44 at least partially with the growth medium 24 prior to the filtration operation. In addition, the housing assembles 14, 16 can be separated to provide access to the filter units 26, 28 positioned therebetween. This is advantageous in that the filter units 26, 28 may be accessed to replace used membrane filters 80, 82 supported on the filter units 26, 28 after one or more filtration cycles.

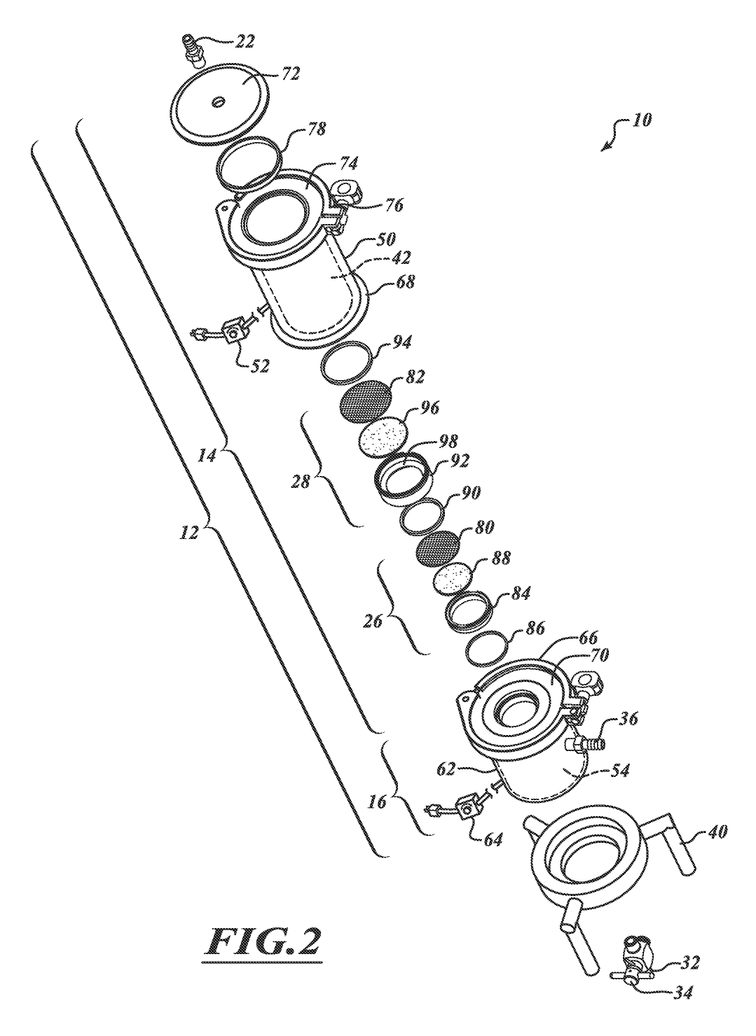

As best shown in FIGS. 2 and 4, the filter units 26, 28 are configured to be stacked between the housing assemblies 14, 16 to provide a sequential series of membrane filters 80, 82 at the interface between the internal cavities 44, 56. More particularly, an upstream filter unit 28 includes a filter cartridge 92 sealingly engaged to a downstream filter unit 26 via an annular seal 90, and which is sealingly engaged to the upper housing assembly 14 via an another annular seal 94. The filter cartridge 92 of the upstream filter unit 28 supports a porous membrane filter support 96 across a central bore thereof. The filter cartridge 92 may include a funnel structure 98 to funnel the growth medium 24 from the upstream filter cartridge 92 toward the downstream filter cartridge 84. The membrane filter support 96 is sufficiently rigid to support the membrane filter 82 thereon and sufficiently porous to enable the growth medium 24 to pass therethrough with relatively less resistance than that of the membrane filter 82 which has a porosity to filter particles of a size greater than a threshold size which is selected from a range of about 0.35 μm to about 1.20 μm. In some embodiments, the membrane filter 82 has a porosity to filter particles of a size greater than a threshold size of about 0.45 μm±0.03 μm. The membrane filter 82 and corresponding support 96 are clamped at an outer edge thereof during assembly and sealingly engage the upper housing assembly 14 via the annular seal 94.

The downstream filter unit 26 includes a filter cartridge 84 sealingly engaged to the lower housing assembly 16 via an annular seal 86. The filter cartridge 84 supports a porous membrane filter support 88 across a central bore thereof. The membrane filter support 88 is sufficiently rigid to support the membrane filter 80 thereon and sufficiently porous to enable the growth medium 24 to pass therethrough with relatively less resistance than that of the membrane filter 80 which has a porosity to filter particles of a size greater than a threshold size which is selected from a range of about 0.10 μm to about 0.40 μm. In some embodiments, the membrane filter 80 has a porosity to filter particles of a size greater than a threshold size of about 0.22 μm±0.03 μm. The membrane filter 80 and corresponding support 88 are clamped at an outer edge thereof during assembly and sealingly engage the upstream filter unit 28 via an annular seal 90. In this manner, the upstream and downstream filter units 26, 28 are sandwiched between the housing assemblies 14, 16 in a substantially sealed manner such that the growth medium 24 is prevented from bypassing the membrane filters 80, 82 during operation of the filtration device 10.

As best illustrated in FIG. 4, the upstream filter unit 28 is configured to position the upstream membrane filter 82 offset from the downstream membrane filter 80 and create a space 100 between the upstream membrane filter 82 and the downstream membrane filter 80 to temporarily receive partially filtered growth medium during the filtration operation. In some embodiments, the upstream membrane filter 82 is offset from the downstream membrane filter 80 by at least one centimeter but less than or equal to ten centimeters. Spacing the membrane filters 80, 82 in this manner advantageously enables multi-stage filtration in a relatively compact form factor but without overly restricting the flow of the growth medium 24. In other embodiments, the membrane filters 80, 82 may be offset from each other by less than one centimeter or more than ten centimeters.

In some embodiments, a ratio of the threshold size of the upstream membrane filter 82 to the threshold size of the downstream membrane filter 80 is between 1.5 and 3.0. In some particularly advantageous embodiments, the threshold size of the upstream membrane filter 82 is about twice the threshold size of the downstream membrane filter 80. For example, in one embodiment, the threshold size of the upstream membrane filter 82 is about 0.45 μm±0.03 μm and the threshold size of the downstream membrane filter 80 is about 0.22 μm±0.03 μm. In this configuration, the filter units 26, 28 may interoperate in a particular efficient manner to sterilize many types of commercially available agar growth mediums, such as, for example, various agar media available from Becton, Dickinson and Company d/b/a BD headquatered in Franklin Lakes, N.J., and Acumedia, a subsidiary of the Neogen Corporation headquartered in Lansing, Mich. Additionally, the growth medium may be a commercially available agar growth medium having a melting temperature at or above about 85° C. or may be an agar, agarose or other growth medium having a relatively lower melting temperature in the range, for example, of about 45° C. to 85° C.

According to some embodiments, in the event the differential in threshold size between the upstream membrane filter 82 and downstream membrane filter 80 is selected to exceed about 0.35 μm, one or more intermediate membrane filters (not shown) having intermediate threshold sizes may be positioned between the other membrane filters 80, 82 to provide a series of membrane filters of sequentially decreasing threshold sizes.

A method of assembling the filtration device 10 will now be described according to the illustrated embodiment shown in FIGS. 1 through 4. First, the stand 40 may be positioned on a support surface. Then, the main body portion 54 and heat source 62 of the lower housing assembly 16 may be positioned on the stand 40 to sit at a height above the support surface. The annular seal 86, which may be, for example, an o-ring, may be positioned within a mouth of the main body portion 54 of the lower housing assembly 16 to provide a sealing interface for the downstream filter unit 26. The downstream filter unit 26 may then be positioned within the mouth of the lower housing assembly 16. The upstream filter unit 28 is in turn positioned on top of the downstream filter unit 26 with an annular seal 90 received therebetween to maintain a fluid tight seal between the filter units 26, 28.

Next, the main body portion 42 and heat source 50 of the upper housing assembly 14 may be positioned over the filter units 26, 28 with another annular seal 94 placed between the upstream filter unit 28 and the main body portion 42 of the upper housing assembly 14 to complete the sealing interface between the filter units 26, 28, and the housing assemblies 14, 16. The housing assemblies 14, 16 may then be clamped or otherwise fastened together with the clamping or fastening device 66 to compress the filter units 26, 28 and seals 86, 90, 94 to form a fluid tight passage extending through the interface between the housing assemblies 14, 16. The lid 72 may be secured to the upper end of the upper housing assembly 14 via the other clamping or fastening device 76 and secured to the pressurized air source 18 via an appropriate conduit (not shown) and fitting 22. The lower end of the lower housing assembly 16 may be fitted with the outlet 32 and discharge valve 34 for selectively discharging sterilized growth medium 24′ from the filtration device 10. Additionally, the lower housing assembly 16 may be fitted with the pressure release fitting 36 and relief valve (not shown) for selectively relieving pressure from the internal cavity 56 of the lower housing assembly 16 during or after a filtration operation.

Although the illustrated embodiment of FIGS. 1 through 4 is shown as including two filter units 26, 28, it is appreciated that in other embodiments, the filtration device 10 may include three, four or more filter units each having a membrane filter of a different porosity stacked together or otherwise presented in a sequential manner. In one embodiment, for example, a filtration device may be provided with three filter units wherein each filter unit has a membrane filter having a porosity to filter particles of a size greater than threshold size which is at least ten percent different from a threshold size of each other membrane filter, and in some embodiments, at least fifty percent different. For instance, in one embodiment, three sequential membrane filters may be provided having threshold sizes of about 0.8 μm, about 0.45 μm, and about 0.22 μm, respectively. In some embodiments, the difference between the threshold size of any two sequential filters is greater than about 0.20 μm but less than about 0.35 μm. In some embodiments, the most upstream positioned membrane filter may have a threshold size between about 0.45 μm and about 1.20 μm and the most downstream positioned membrane filter may have a threshold size between about 0.10 μm and about 0.25 μm. When the differential in threshold size between the most upstream positioned membrane filter and the most downstream positioned membrane filter exceeds about 0.35 μm, at least one intermediary membrane filter of an intermediate threshold size is beneficial to maintain an efficient filtration mechanism.

Furthermore, although the illustrated embodiment shown in FIGS. 1 through 4 is shown as including two separately coupleable housing assemblies 14, 16, it is appreciated that in other embodiments, three or more housing assemblies may be coupled together in a similar fashion with one or more filter units 26, 28 positioned therebetween to form a three-stage, four-stage or other multi-stage filtration device for various growth mediums, including, for example, agar growth mediums.

A method of filtering a growth medium will now be described, according to one embodiment. The method may begin by preparing a growth medium in the form of an agar growth medium 24, for example. The preparation procedure may include stirring dehydrated agar culture media with distilled water and heating the mixture to 45° C. or above or until it boils to form a heated, flowable agar growth medium 24. The growth medium may be a commercially available agar growth medium having a melting temperature at or above about 85° C. or may be an agar, agarose or other growth medium having a relatively lower melting temperature in the range, for example, of about 45° C. to 85° C. The method may continue by depositing the agar growth medium 24 in an internal cavity 44 of an upper housing assembly 14 of a filtration device 10. The growth medium 24 may be deposited in the internal cavity 44 through an open end of the upper housing assembly 14 in which a lid 72 has been removed or through any other input mechanism or device (e.g., feed screw, mechanical gate, etc.) that may be selectively sealed once the agar growth medium 24 has been deposited. Prior to depositing the agar growth medium 24, a heat source 50 may be activated to begin an optional pre-heating process. The method may continue by heating the agar growth medium 24 within the internal cavity 44 of the filtration device 10 to maintain the growth medium above a gelling temperature or melting temperature thereof. For example, in some embodiments the method may continue by heating the agar growth medium 24 within the internal cavity 44 of the filtration device 10 to 45° C. or more and/or maintaining the agar growth medium 24 at or above 45° C. as the agar growth medium 24 is subsequently filtered. In other embodiments, the agar growth medium 24 may be heated to and/or maintained at or above 85° C. as the agar growth medium 24 is subsequently filtered. In still other embodiments, the agar growth medium 24 may be heated to and/or maintained between about 85° C. and about 100° C., and in still other embodiments, may be heated to and/or maintained between about 90° C. and about 100° C. as the agar growth medium 24 is subsequently filtered. The desired temperature of the agar growth medium 24 for the filtration process may depend on several factors including, for example, the type of agar used and the presence or absence of certain additives. In some embodiments, the method may also include preparing the agar growth medium 24 with alcohol or another solvent to extract low molecular weight agar to affect the temperature at which the agar growth medium may be heated and/or maintained.

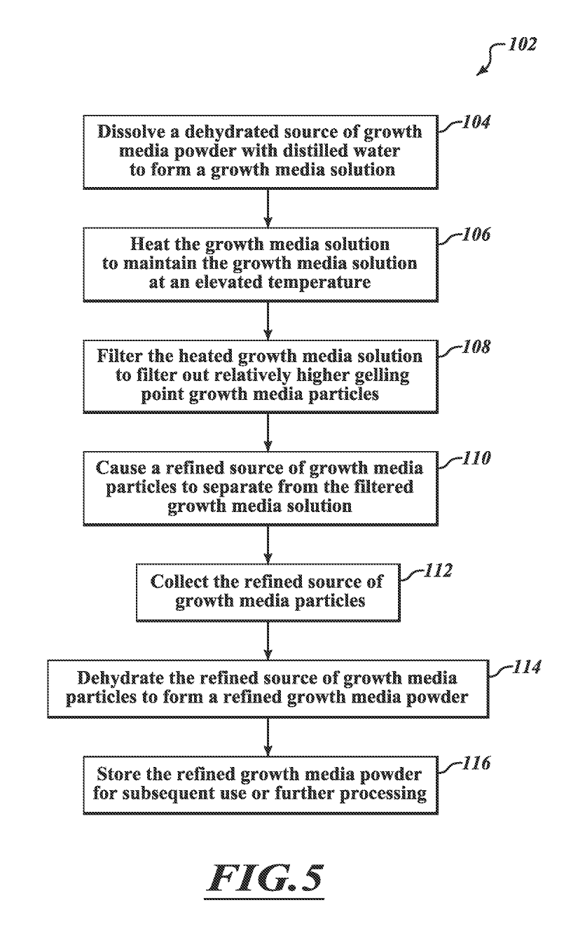

According to some embodiments, a commercially available growth media or other initial source of growth media may be mechanically filtered to isolate low gelling point and low molecular weight particles to form a refined source of growth media having a low gelling point and low molecular weight characteristic which is particularly well adapted to be filter sterilized by the devices described herein under relatively low temperature conditions. For example, FIG. 5 shows a process flow diagram for a method 102 of preparing a refined source of growth media powder, according to one example embodiment, which is particularly well adapted to be filter sterilized.

The method 102 begins at 104 where a dehydrated source of growth media powder, such as, for example, an agar nutrient powder, is dissolved with distilled water to form a growth media solution. At 106, the growth media solution is heated to maintain the growth media solution at an elevated temperature to maintain the growth media solution in a flowable form. As an example, in some embodiments, heating the growth media solution at 106 may include heating an agar nutrient solution to between about 45° C. and 65° C. to maintain the agar nutrient solution in a liquid, flowable form for subsequent processing. Heating of the growth media solution at 106 may occur simultaneously with the dissolving of the dehydrated source of growth media powder at 104.

Next, at 108, the heated growth media solution may be passed through one or more filter devices to filter out relatively higher gelling point and higher molecular weight growth media particles. For instance, in some embodiments, a heated agar nutrient solution may be passed through a series of membrane filter elements having pore sizes in the range of between about 0.10 μm and 0.65 μm to extract or filter out relatively higher gelling point and higher molecular weight growth media particles. In one particularly advantageous embodiment, a heated agar nutrient solution may be passed through two sequential membrane filter elements having pore sizes of about 0.45 μm and about 0.20 μm, respectively. According to some embodiments, the filtration devices 10 described herein may be utilized to filter the growth media solution at 108 to filter out relatively higher gelling point and higher molecular weight growth media particles.

At 110, the method 102 continues with causing a refined source of growth media particles, such as, for example, agar media particles, to separate from the filtered growth media solution. For example, in some embodiments, causing the refined source of growth media particles to separate from the filtered growth media solution at 110 may include an ethanol precipitation process or polyethylene glycol precipitation process. More particularly, a 95% ethanol or a polyethylene glycol (PEG) solution may be added to the filtered growth media solution for an extended period of time (e.g., 24 hours) to form a precipitate of solid growth media particles from the solution. The solid growth media particles may then be collected at 112 by passing the solution through a filter element to isolate the solidified agar media particles or by other separation techniques.

At 114, a refined source of growth media particles can then be dehydrated to form a refined growth media powder which may be stored or packaged for subsequent use at 116. Dehydrating the refined source of growth media particles at 112 may include, for example, vacuum heating a refined source of agar media particles at a temperature between about 60° C. and 80° C. Storing the refined growth media powder at 116 may include storing a refined agar nutrient powder in a container or package for subsequent use or transport.

The resulting growth media powder of the method 102 described above is advantageously characterized by a relatively lower gelling point and lower molecular weight when combined with distilled water in a gelatinous form. In this manner, the growth media powder may be combined with distilled water and maintained at an elevated temperature that is relatively lower when compared to unrefined growth media powders when filter sterilizing the resulting growth medium according to the filter sterilizing methods described herein. In some embodiments, for example, the gelling temperature may be reduced by ten degrees, twenty degrees or more. This is particularly advantageous in enabling filter sterilization under relatively low temperature conditions which may facilitate increased processing speeds as well as substantially prevent degradation that might otherwise occur due to exposure to higher temperature conditions. In addition, according to some embodiments, the resulting growth media powder of the method 102 may enable sufficient sterilization to occur when filter sterilizing the refined growth media with a growth medium filtration device having only a single membrane filter.

Irrespective of the gelling point characteristics of the growth medium, the growth medium 24 is raised to and/or maintained at a temperature sufficient to establish the growth medium 24 in a flowable form so that it may pass with relative ease through a series of membrane filters 80, 82. In some embodiments, an agar growth medium 24 may be filtered at a rate of about 75 ml/min. Accordingly, a sample of about 300 ml of sterilized agar medium 24′ may be prepared in about four minutes. This is significantly faster than other conventional methods of preparing sterilized agar, such as, for example, by autoclave sterilization or gamma sterilization.

Next, a pressurized air source 18 may be controlled to pressurize the internal cavity 44 and force the agar growth medium 24 to pass sequentially through an upstream filter unit 28 supporting an upstream membrane filter 82 which has a porosity to filter particles of a size greater than a threshold size selected from a range of about 0.35 μm to about 1.20 μm and then through a downstream filter unit 26 supporting a downstream membrane filter 80 which has a porosity to filter particles of a size greater than a threshold size selected from a range of about 0.10 μm to about 0.40 μm but less than the threshold size of the upstream membrane filter 82. In this manner, relatively larger impurities are filtered in a first stage of filtration via the upstream membrane filter 82 and relatively smaller impurities are filtered in a second stage of filtration via the downstream membrane filter 80 to provide a sterilized agar growth medium 24′ to meet generally accepted levels of purity. For example, in some embodiments, the sterilized growth medium 24′ is essentially free of any impurities over 0.22 μm upon completion of the sterilization process.

According to some embodiments, in the event the differential in threshold size between the upstream membrane filter 82 and downstream membrane filter 80 exceeds about 0.35 μm, one or more intermediate membrane filters (not shown) having intermediate threshold sizes may be positioned between the other membrane filters 80, 82 to provide a series of membrane filters of sequentially decreasing threshold sizes.

After sterilization, the sterilized growth medium 24′ may be stored in the internal cavity 56 of a lower housing assembly 16 positioned downstream of the filter units 26, 28, or discharged from the filtration device 10 through an outlet 32 by actuating a discharge valve 34. Pressure built up inside the housing 12 during operation may be relieved via a pressure relief valve coupled to the internal cavity 56 of the lower housing assembly 16. The pressure may be relieved after the filtering process but prior to discharging the sterilized growth medium 24′ from the filtration device 10 such that the sterilized growth medium 24′ may be stored in an unpressurized state.

According to some embodiments, the sterilized growth medium 24′ may then be intermittingly discharged from the filtration device 10 into a series of Petri dishes. Appropriate control systems and sequencing mechanisms may be provided to intermittingly discharge the sterilized growth medium 24′ into the Petri dishes in an automated manner. Alternatively, the sterilized growth medium 24′ may be discharged into the Petri dishes to form agar plates by manually actuating the discharge valve 34. Once prepared, the agar plates can be used for culturing microorganisms in a conventional manner.

Although embodiments of the methods described above include a dual stage filtration process sufficient to sterilize many commercially available agar and other gelatinous substance based growth mediums, it is appreciated that in some embodiments, a multistage filtration process which includes passing an agar growth medium or other similar growth medium through three, four or more membrane filters may be provided. In other embodiments, it is appreciated that a single stage filtration process which includes passing a refined agar growth medium or other similar growth medium through only one membrane filter may be provided. In addition, although the method described above includes passing growth medium 24 from an upper housing assembly 14 to a lower housing assembly 16, it is appreciated that in other embodiments, the method may include passing the growth medium through one or more intermediate housings positioned between the upper housing assembly 14 and the lower housing assembly 16.

Moreover, the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A method of filtering a growth medium, the method comprising:
maintaining the growth medium at an elevated temperature within an internal cavity of a filtration device upstream of both of an upstream filter unit and a downstream filter unit, the upstream filter unit configured to support a upstream membrane filter having a porosity to filter particles of a size greater than a first threshold size and the downstream filter unit configured to support a downstream membrane filter having a porosity to filter particles of a size greater than a second threshold size that is smaller than the first threshold size; and
pressurizing the internal cavity to assist in forcing the growth medium to pass sequentially through the upstream filter unit and the downstream filter unit to sterilize the growth medium for use in culturing microorganisms.
